SEQUENCE LISTING (7 sequences)

SEQ ID NO: 1 - protein, 555 aa, Dermatophagoides farinae
  MISC_FEATURE: gi5815436 Der f 15
MKTIYAILSIMACIGLMNASIKRDHNDYSKNPMRIVCYVGTWSVYHKVDPYTIEDIDPFKCTHLMYGFAKIDEYKYTIQVFDPYQDDNHNSWEKRGYERFNNLRLKNPELTTMISLGGWYEGSEKYSDMAANPTYRQQFIQSVLDFLQEYKFDGLDLDWEYPGSRLGNPKIDKQNYLALVRELKDAFEPHGYLLTAAVSPGKDKIDRAYDIKELNKLFDWMNVMTYDYHGGWENFYGHNAPLYKRPDETDELHTYFNVNYTMHYYLNNGATRDKLVMGVPFYGRAWSIEDRSKLKLGDPAKGMSPPGFISGEEGVLSYIELCQLFQKEEWHIQYDEYYNAPYGYNDKIWVGYDDLASISCKLAFLKELGVSGVMVWSLENDDFKGHCGPKNPLLNKVHNMINGDEKNSFECILGPSTTTPTPTTTPTTPTTTPTTPSPTTPTTTPSPTTPTTTPSPTTPTTTPSPTTPTPTTPTPAPTTSTPSPTTTEHTSETPKYTTYVDGHLIKCYKEGDIPHPTNIHKYLVCEFVNGGWWVHIMPCPPGTIWCQEKLTCIGE

SEQ ID NO: 2 - protein, 462 aa, Dermatophagoides farinae
  MISC_FEATURE: gi27550039 Der f 18
MTRFSLTVLAVLAACFGSNIRPNVATLEPKTVCYYESWVHWRQGEGKMDPEDIDTSLCTHIVYSYFGIDAATHEIKLLDEYLMKDLHDMEHFTQHKGNAKAMIAVGGSTMSDQFSKTAAVEHYRETFVVSTVDLMTRYGFDGVMIDWSGMQAKDSDNFIKLLDKFDEKFAHTSFVMGVTLPATIASYDNYNIPAISNYVDFMNVLSLDYTGSWAHTVGHASPFPEQLKTLEAYHKRGAPRHKMVMAVPFYARTWILEKMNKQDIGDKASGPGPRGQFTQTDGFLSYNELCVQIQAETNAFTITRDHDNTAIYAVYVHSNHAEWISFEDRHTLGEKAKNITQQGYAGMSVYTLSNEDVHGVCGDKNPLLHAIQSNYYHGVVTEPTVVTLPPVTHTTEHVTDIPGVFHCHEEGFFRDKTYCATYYECKKGDFGLEKTVHHCANHLQAFDEVSRTCIDHTKIPGC

SEQ ID NO: 3 - protein, 14 aa, Dermatophagoides farinae
  MISC_FEATURE: Der f 15 835.38
FDGLDLDWEYPGSR

SEQ ID NO: 4 - protein, 13 aa, Dermatophagoides farinae
  MISC_FEATURE: Der f 15 776.38
VDPYTIEDIDPFK

SEQ ID NO: 5 - protein, 14 aa, Dermatophagoides farinae
  MISC_FEATURE: Der f 15 813.89
IWVGYDDLASISCK

SEQ ID NO: 6 - protein, 17 aa, Dermatophagoides farinae
  MISC_FEATURE: Der f 18 505.99
TVHHCANHLQAFDEVSR

SEQ ID NO: 7 - protein, 7 aa, Dermatophagoides farinae
  MISC_FEATURE: Der f 18 383.19
GDFGLEK

(19) United States
(12) Patent Application Publication
(10) Pub. No.: US 2021/0085782 A1
(43) Pub. Date: Mar. 25, 2021

(54) VETERINARY PRODUCT

(71) Applicant: LABORATORIOS LETI S.L. UNIPERSONAL, Madrid (ES)

(72) Inventors: Jeronimo Carnes SANCHEZ, Madrid (ES); Raquel Moya LOBO, Madrid (ES); Laura Ramio LLUCH, Madrid (ES); Pilar Brazis CAUBET, Madrid (ES); Anna Puigdemont RODRIGUEZ, Barcelona (ES)

(21) Appl. No.: 16/635,277

(22) PCT Filed: Jul. 30, 2018
(86) PCT No.: PCT/EP2018/070637

§ 371 Date: Jan. 30, 2020

(30) Foreign Application Priority Data
Jul. 31, 2017 (EP) 17382527.4

(51) Int. Cl. A61K 39/35

(52) U.S. Cl. CPC A61K 39/35; A61K 2039/54

(57) ABSTRACT

The present invention relates to a purified veterinary allergen extract enriched. In particular, the invention relates to a purified veterinary allergen extract enriched with Der f 15 and Der f 18. The invention further relates to use of the allergen extract as a veterinary product, and its use in treating allergy, in particular house dust mite allergy in mammals, more particularly, dogs.

### FIELD OF THE INVENTION

The present invention relates to a purified veterinary allergen extract enriched. In particular, the invention relates to a purified veterinary allergen extract enriched with Der f 15 and Der f 18. The invention further relates to use of the allergen extract as a veterinary product, and its use in treating allergy, in particular house dust mite allergy in mammals, more particularly, dogs.

### BACKGROUND OF THE INVENTION

Atopic dermatitis is a pruritic allergic skin disease that affects approximately 10% of dogs and is related to the production of IgE antibodies against environmental allergens. The main cause of non-seasonal allergies in dogs is house dust mites (HDM), in particular the species Dermatophagoides farinae. Allergic dogs are treated with drugs for controlling the symptoms, but alternative treatments include Specific Immunotherapy (SIT) which involves the administration of increasingly larger doses of an allergen extract with the aim of inducing immunological tolerance. Allergen immunotherapy modulates the immune response to the allergen rather than ameliorating the symptoms induced by an allergic reaction, and can either reduce the need for medication, reduce the severity of symptoms or eliminate hypersensitivity altogether.

Relevant differences have been reported regarding the human and canine HDM allergens profile. Whereas the major allergens for humans (Der f 1 and Der f 2) are proteins of relatively low molecular weight, the most important allergens of D. farinae in atopic dogs (Der f 15 and Der f 18) are included in groups 15 and 18, belonging to the high molecular weight fraction of mites. Despite these differences, allergen specific immunotherapy for dogs has always been prepared directly with allergen extracts developed and characterised for human immunotherapy. One problem with treating dogs with allergen extracts developed and characterised for human immunotherapy is that the dogs may well become sensitized to previously non-offending allergens such as Der f 1 and Der f 2.

There is a need therefore for the development of a specific immunotherapy against house dust mites for veterinary use, in particular for dogs.

### SUMMARY OF THE INVENTION

The present inventors have developed a veterinary mite extract enriched with high molecular weight fractions. This fraction contains the allergens recognized by serum samples from mite sensitized dogs.

In a first aspect of the invention, there is provided a veterinary mite extract enriched with proteins with molecular weights higher than 50 kDa.

In one embodiment, the veterinary mite extract is enriched with the allergens Der f 15 and Der f 18 relative to human extract.

In one embodiment, the extract has low levels of allergens Der f 1 and Der f 2 relative to human extract.

In a further aspect of the present invention, there is provided a process for producing a veterinary mite allergen extract comprising:
a) contacting a source material comprising a veterinary mite allergen (raw material) with an allergen extract agent to produce a mixture of allergens dissolved in liquid phase, and a solid phase comprising non-allergenic residue;
b) subjecting the mixture to a separation step to isolate the allergens dissolved in the liquid phase, to produce a crude allergen extract;
c) subjecting the crude allergen extract to a medium molecular fraction removal step to remove molecules having a size of less than 50 kDa and applying a pressure of 1.8 bar during the removal step;
d) carrying out step c) at 3-5° C. until the allergen extract has conductivity of below 1050 μS/cm, as measured at room temperature, and/or until a specific volume of distilled water has been used, to obtain a purified native allergen extract.

According to a third aspect of the invention, there is provided a veterinary mite allergen extract for use as an active therapeutic substance in the treatment of allergy.

### BRIEF DESCRIPTION OF THE FIGURES

FIG. 1 shows SDS-PAGE of 10 μg protein of D. farinae extracts: low range MW standard (1); human extract (2); Extract 1 (3); Extract 2 (4); and Extract 3 (5).

FIG. 2 shows SDS-PAGE in Any kD TGX gels of 10 μg protein of D. farinae extracts: HiMark prestained marker (LifeTechnologies) (1); human extract (2); Extract 1 (3); Extract 2 (4); Extract 3 (5); and broad range MW standard (Bio-Rad) (6).

FIG. 3 shows SDS-PAGE of 5 μl of D. farinae extracts after cleaning procedure: low range MW standard (1); Extract 1 (2); Extract 2 (3); and Extract 3 (4).

FIG. 4 shows 2D of 170 μg protein of D. farinae: Extract 1 (A); Extract 2 (B); Extract 3 (C); human extract (D); low range MW standard is indicated in each gel.

FIG. 5 shows chromatographic profiles of D. farinae extract: human extract (A); Extract 1 (B); Extract 2 (C); and Extract 3 (D).

FIG. 6 shows sequenced peptides for Der f 15 (A, C, E) (SEQ ID NO. 1) and Der f 18 (B, D, F) (SEQ ID NO. 2) from Extract 1 (A, B), Extract 2 (C, D) and Extract 3 (E, F) veterinary extracts.

FIG. 7 shows immunoblot of 10 μg protein of D. farinae extracts: low range MW standard (1); human extract (2); Extract 1 (3); Extract 2 (4); and Extract 3 (5).

FIG. 8 shows specific IgE optical density of a pool of dogs sera incubated with human (PRI 6756LN batch) and veterinary (080616LN, 090616LN, 290616LN for each of extracts 1, 2 and 3 respectively) D. farinae extracts.

FIG. 9 shows specific IgE optical density of individual serum samples against veterinary D. farinae extract 4 and human D. farinae extract.

FIG. 10 shows an ELISA potency assay for veterinary D. farinae extract 4 (sample by duplicate).

FIG. 11 shows an ELISA potency assay for human D. farinae extract and veterinary D. farinae extract 4.

FIG. 12 shows an immunoblot image analyzed by ImageQuant. D. farinae extracts: veterinary extract 4 (2) human extract (3), and veterinary extract 2 (4). 1: Low range MW standard (kDa). Bands corresponding to Der f 15 and Der f 18 are marked.

FIG. 13 shows an immunoblot image analyzed by ImageQuant. D. farinae extracts: veterinary extract 5 (2) veterinary extract 4 (3), and human extract (4). 1: Low range MW standard (kDa). Bands corresponding to Der f 15 and Der f 18 are marked.

FIG. 14 shows bands identified in immunoblot images from FIG. 12 (left image) and FIG. 13 (right image).

FIG. 15 shows immunoblot of 10 μg protein of the human D. farinae extract and veterinary D. farinae extract 4. STD: low range MW standard. A) positive sera 1, 2; B) positive sera 3, 4 and negative control C1; C) positive sera 5-7 and negative control C2.

FIG. 16 shows IL-10 and IFN-γ produced by Peripheral Blood Mononuclear Cells (PBMCs) from atopic and control groups after 24 (IL-10) or 48 (IFN-γ) hours of incubation with negative control, human extract or veterinary extract 2 (significant differences (p value) are indicated in each graph showing median value and interquartile range).

### DETAILED DESCRIPTION

The process described herein yields a veterinary mite allergen extract which exhibits increased IgE binding of serum samples from mammals suffering atopic dermatitis caused by D. farinae.

The allergen extracts of the invention are derived from any source material comprising mite allergens which are known to illicit an IgE mediated immune reaction in an animal. In particular, the source material is a mite allergen which is known to illicit an IgE mediated immune reaction in a dog.

In a first aspect of the invention, there is provided a veterinary mite extract enriched with proteins with molecular weights greater than 50 kDa.

In one embodiment, the veterinary mite extract is derived from D. farinae.

In one embodiment, the veterinary mite extract is enriched with the allergens Der f 15 and Der f 18 relative to human extract. Allergens Der f 15 and Der f 18 are the major allergens of D. farinae in atopic dogs, and may also be important allergens in other mite species, for example Dermatophagoides pteronyssinus.

In one embodiment, the extract has lower levels of allergens Der f 1 and Der f 2 relative to human extract. The level of allergens present in the extract can be determined from SDS by densitometry analysis. For Der f 1 and Der f 2, these allergens can also be quantified by commercial kits such as those available from Indoor Biotechnologies Inc (Charlottesville, Va., US).

The term “enriched” means a veterinary mite allergen extract with a higher concentration of proteins with molecular weights greater than 50 kDa when compared to human extract, in particular, with higher levels of the allergens Der f 15 and Der f 18, as determined from SDS by densitometry analysis.

The term “human extract” means an extract which is obtained from steps a) to d) disclosed herein, but where step c) removes molecules having a size of less than 3 kDa by using a 3 kDa molecular weight cut-off dialysis membrane and an approximately pressure of only 1 bar is applied.

In one embodiment, in a 50% inhibition ELISA IgE assay, the veterinary mite allergen extract of the first aspect of the invention is at least 1.5 times, preferably at least 2.0 times, most preferably 2.0-2.5 times more effective than human extract.

In another embodiment, in an immunoblot densitometry analysis, in the veterinary mite allergen extract, the concentration of Der f 15 is at least 1.5 times, preferably at least 2.0 times, most preferably 2.0-3.0 times higher than human extract, and/or wherein the concentration of Der f 18 is at least 1.5 times, preferably at least 2.0 times, most preferably 2.0-3.5 times higher than human extract.

In yet another embodiment, the veterinary mite allergen extract comprises recombinant Der f 15 and Der f 18, preferably the recombinant Der f 15 and Der f 18 has at least 70%, more preferably at least 80%, even more preferably at least 90% homology with non-recombinant Der f 15 and Der f 18 respectively, and the veterinary mite allergen extract up-regulates expression of IFN-γ and IL-10 in peripheral blood mononuclear cells.

Recombinant proteins, in accordance with the invention, may be expressed using a protein expression system, such as a bacterial, yeast, insect, plant, avian or mammalian expression system. Suitable systems are familiar to those skilled in the art. Protein expression and purification is carried out as known to those skilled in the art, for example as outlined in Structural Genomics Consortium et al., “Protein Production and Purification”, Nature methods, 5, 2, 135 (2008). In some embodiments, the proteins may be glycosylated.

In a further aspect of the present invention, there is provided a process for producing a veterinary mite allergen extract comprising:
a) contacting a source material comprising a mite allergen with an allergen extract agent to produce a mixture of allergens dissolved in liquid phase, and a solid phase comprising non-allergenic residue;
b) subjecting the mixture to a separation step to isolate the allergens dissolved in the liquid phase, to produce a crude allergen extract;
c) subjecting the crude allergen extract to a medium molecular fraction removal step to remove molecules having a size of less than 50 kDa and applying a pressure of 1.8 bar during the removal step;
d) carrying out step c) at 3-5° C. until the allergen extract has conductivity of below 1050 μS/cm and/or until a specific volume of distilled water has been used, to obtain an enriched allergen extract.

The source material may be selected from family Pyroglyphidae, which includes the species oki Dermatophagoides farinae, Dermatophagoides pteronyssinus and Euroglyphus maynei, and/or family Acaridae.

In one embodiment the source material is Dermatophagoides farinae.

In a preferred embodiment of the invention the source material is a mite culture with >80% of D. farinae bodies. The remaining percentage of the source material may comprise mite faeces and/or culture medium. The percentage of mite bodies can be determined by observing the mite culture under a microscope and counting the number of mite bodies relative to other particles in a mite culture.

Allergens are obtained from the source material by extraction with an allergen extract agent to produce a crude allergen extract comprising allergens dissolved in liquid phase and a solid phase comprising “unwanted” non-allergenic residue. The allergen extract agent may be an aqueous solution, and preferably comprises a buffering agent. The allergen extraction agent may comprise PBS and/or NaCl, for example a solution of 0.01 M PBS/0.15 M NaCl, or ammonium bicarbonate (NH₄)HCO₃ and/or NaCl, for example a solution of 0.125 M (NH₄)HCO₃/0.15 M NaCl. The source material may be extracted in the allergen extract agent in any ratio where the weight of the allergen extract agent exceeds the weight of the source material, for example 1:2, 1:3, 1:5, 1:10, 1:20, 1:50, 1:80. Preferably, the source material is extracted in the allergen extract agent in a ratio of 1:10 source material:allergen extract agent (wt/wt). The ratio of the source material to allergen extract agent in the extraction step (step a) may vary but should be such that the allergens in the source material residue can dissolve in the allergen extract agent. The extraction of the source material with the allergen extract agent is preferably performed for sufficient time for the allergens in the source material residue to dissolve in the allergen extraction agent, which may be for between 30 minutes to 12 hours, preferably between 1 to 6 hours, more preferably between 2 to 5 hours, and most preferably for around 4 hours. The allergen extraction step may be performed at between 20 to 25° C., but is preferably performed cold at between 2 to 6° C., and most preferably between 3 to 5° C. During the allergen extraction step, the source material is preferably stirred or agitated with the allergen extraction agent.

After the allergen extraction step, the allergens dissolved in the liquid phase are separated from the source material residue, to produce a crude allergen extract. The separation step is preferably centrifugation, although many techniques to separate solid from liquid are applicable, these being well known to a person skilled in the art. Preferably, the allergens dissolved in liquid phase are centrifuged at between 2 to 6° C., and preferably between 3 to 5° C., for sufficient time to sediment the source material residue as pellet, for example between 1 minute to 1 hour, or over 1 hour. The crude allergen extract (i.e. the supernatant containing the dissolved allergens) may be stored at between 2 to 6° C. The source material residue pellet may be further extracted with the allergen extract agent using the same conditions as the first allergen extraction step (step a), and preferably for a longer extraction period such as between 4 to 8 hours, 8 to 12 hours, or over 12 hours. After the second allergen extraction step, the allergens dissolved in liquid phase may be separated from the source material residue to produce a crude allergen extract. The crude allergen extracts from the first and second allergen extraction steps are preferably pooled for further treatment.

The crude allergen extract may be filtered, for example using filters with a 0.8-1.2 μm pore size. The crude allergen extract is then subjected to a medium molecular fraction removal step to remove molecules having a medium molecular size such as low molecular weight allergens, salts and other non-allergenic compounds. In step c) molecules having a molecular size of less than 50 kDa may be removed.

During the medium molecular fraction removal step, a pressure is fixed between 1.2 and 1.8 bar. The step of applying a pressure of about 1.2 to 1.8 bar during the process of medium molecular fraction removal is important to the present invention. Applying increased pressure, compared to prior art extraction methods at 1 bar, improves the efficacy of the process. Preferably, the pressure is fixed at 1.8 bar.

The medium molecular fraction removal step is preferably continued at 3 to 5° C. until the conductivity of the allergen extract is less than 1050 μS/cm, or less than 900 μS/cm, or less than 800 μS/cm, or less than 700 μS/cm, or less than 600 μS/cm, or preferably, less than 500 μS/cm (measured at room temperature). In addition, or alternatively, the medium molecular fraction removal step is continued until a specific volume of distilled water has been used. A suitable volume of distilled water necessary for the diafiltration can be calculated using the following formula: ml filtrated extract×10=distilled water volume. The result is a purified native allergen extract enriched with higher molecular weight proteins.

The medium molecular weight removal step may comprise an ultrafiltration step, a diafiltration step, a dialysis step, or filtration. Preferably, the medium molecular weight removal step (step c) comprises a diafiltration step.

The resulting native allergen extract may be filtered, for example using a 0.22 μm pore size, and may be frozen or freeze dried for storage.

The present invention further comprises a treatment for veterinary mite allergy and a diagnostic extract for veterinary mite allergy, both comprising allergen extracts produced by the processes of the present invention, as the active ingredient. The veterinary mite allergy may be associated with exposure to mite allergens such as D. farinae which illicit an IgE mediated allergic response as discussed herein.

According to a further aspect of the present invention there is provided a veterinary mite allergen extract obtained by or obtainable according to the processes described herein.

The veterinary mite allergen extract may be for use in the treatment of mite allergy in animals. In a preferred embodiment the allergen extract may be for use in the treatment of mite allergy in dogs. In another preferred embodiment the allergen extract may be for use in the treatment of mite allergy in a cat, wherein the cat comprises serum IgE antibodies which in an immunoblot assay react with Dermatophagoides farinae allergens with molecular weights higher than 40 kDa and including Der f 15 and Der f 18 or recombinant Der f 15 and Der f 18, preferably the recombinant Der f 15 and Der f 18 has at least 70%, more preferably at least 80%, even more preferably at least 90% homology with non-recombinant Der f 15 and Der f 18 respectively.

The allergen extract of the present invention may be characterised by the following physicochemical and biological properties:
i. Increased protein content, determined by Bradford method, with respect to the D. farinae human extract;
ii. Reduction of protein content with a molecular weight lower than 30 kDa, identified as bands by SDS-PAGE in reducing conditions, with respect to the human extract, except for a band of about 20 kDa, that it is still present in the veterinary extract;
iii. Reduction in Der f 1 (about 30 kDa) and Der f 2 (about 15 kDa) content with respect to the human extract, as determined by quantification ELISA kits from Indoor;
iv. Reduction of IgE recognition bands with a molecular weight lower than 30 kDa, identified by immunoblot in reducing conditions, except for a band of about 20 kDa, compared to the human extract;
v. Modification in the molecular weight (MW) distribution profile with respect to the human extract, determined by size-exclusion chromatography;
vi. Increase in the IgE binding with respect to the human extract, determined by IgE ELISA experiments using a specific pool of sera from sensitized individuals;
vii. Presence of dog major allergens Der f 15 and Der f 18, identified by mass-spectrometry;
viii. Increase in Der f 15 content with respect to the human extract, determined by relative protein quantification by targeted proteomics;
ix. Increase in Der f 15 and Der f 18 content with respect to the human extract, determined by immunoblot densitometry analysis using a specific pool of sera from sensitized individuals;
x. Increase in biological potency with respect to the human extract, determined by an ELISA 50% inhibition assay using a specific pool of sera from sensitized individuals;
xi. Induction of the production of IL-10 and IFN-γ cytokines in Peripheral Blood Mononuclear Cells from dogs sensitized to D. farinae.

The allergen extracts of the present invention may be for use as an active component of a medicament for the treatment of an allergic animal, with the aim of inducing tolerance to certain mite allergens.

There is provided the use of an allergen extract according to the present invention in diagnostics for immunological disorders, preferably to detect allergic disease. There is provided the use of an allergen extract according to the present invention for the treatment of mite allergy or in the manufacture of a medicament for the treatment of mite allergy. The use may be for immunotherapy. The use may be for standardisation, diagnosis, synthesis and vaccination purposes. The use may be in therapeutic treatment of animals, preferably in immunotherapy. The use may be in monitoring the animals during immunotherapy.

According to a further aspect of the present invention there is provided a pharmaceutical composition comprising an allergen extract according to the present invention. There is also provided a pharmaceutical composition for the treatment of mite allergy which comprises as the active ingredient a pharmaceutically effective amount of a veterinary mite allergen extract according to the present invention and at least one pharmaceutically acceptable carrier or diluent. There is provided a diagnostic composition for mite allergy which comprises as the active ingredient a diagnostically effective amount of an allergen extract according to the present invention.

According to a further aspect of the present invention there is provided a vaccine comprising a veterinary mite allergen extract according to the present invention. The pharmaceutical composition and vaccine may further comprise one or more adjuvants, diluents, preservatives or mixtures thereof. The pharmaceutical composition or vaccine may comprise a physiologically acceptable carrier. As used herein, the phrase “pharmaceutically acceptable” preferably means approved by a regulatory agency of a government, or listed in the European or US Pharmacopeia or another generally recognised pharmacopeia for use in animals.

Such pharmaceutically acceptable carriers can be sterile liquids, such as water and oils, including those of petroleum, animal, vegetable or synthetic origin, such as peanut oil, soybean oil, mineral oil, sesame oil and the like. Saline solutions and aqueous dextrose and glycerol solutions can also be employed as liquid carriers, particularly for injectable solutions. Suitable pharmaceutical excipients include mannitol, starch, glucose, lactose, sucrose, gelatin, malt, rice, flour, chalk, silica gel, magnesium carbonate, magnesium stearate, sodium stearate, glycerol monostearate, talc, sodium chloride, dried skim milk, glycerol, propylene, glycol water, ethanol and the like.

There is provided a vaccine obtainable according to the process of the first aspect of the present invention. The vaccine may be for sub-cutaneous, sub-lingual or epicutaneous use.

There is provided the use of a vaccine according to the present invention in the treatment of mite allergy or in the manufacture of a medicament for the treatment of mite allergy.

According to a further aspect of the present invention there is provided a method of preventing an allergen sensitisation comprising the step of: exposing an animal to an effective amount of an allergen extract, the pharmaceutical composition or the vaccine of the present invention.

According to a further aspect of the present invention there is provided a method of treating a mite allergy in a sensitised mammal, comprising administering to the mammal an effective amount of an allergen extract, the pharmaceutical composition or the vaccine of the present invention. The allergen extract, the pharmaceutical composition or the vaccine may be administered subcutaneously or sublingually, and may be administered as an increasing or constant dosage. The term “mammal” excludes human beings.

Preferably the mammal is a dog. Preferably the mammal is a cat, wherein the cat comprises serum IgE antibodies which in an immunoblot assay react with Dermatophagoides farinae allergens with molecular weights higher than 40 kDa and including Der f 15 and Der f 18 or recombinant Der f 15 and Der f 18, preferably the recombinant Der f 15 and Der f 18 has at least 70%, more preferably at least 80%, even more preferably at least 90% homology with non-recombinant Der f 15 and Der f 18 respectively.

The present invention is illustrated by the following examples which detail processes for the preparation of the extracts comprising allergens.

### Examples

The source material, which may also be referred to as the raw material, used for the extract production was a mite culture with >80% of D. farinae bodies cultivated by Laboratorios LETI and is commercially available. The three veterinary extracts were prepared according to the following method:

A. Extraction I
The raw material to be extracted was weighed and the weight noted down;
The volume of extracting agent (PBS 0.01M-NaCl 0.15M) required was calculated using the following formula:

Raw material (g)×20=ml extracting agent;
The raw material was placed in a container with half of the extracting agent volume calculated in the previous step and was extracted at 3-5° C. with magnetic stirring for 4 hours:

ml extracting agent (total vol.)÷2=ml extracting agent (Volume I);
The mixture was centrifuged at 10,000 rpm at 5° C. for at least 30 minutes;
The supernatant was then recovered and the volume was noted down. The supernatant was kept at 3-5° C. in a labelled closed container. Care was taken to avoid the rest of the pellet from going in the supernatant, to ensure maximum clarity.

B. Extraction II
The pellet from extraction I was added to a container with the rest of the extracting agent volume (Volume II). The mixture was extracted with agitation at 3-5° C. for at least 8 hours;
The mixture was then centrifuged at 10,000 rpm at 5° C. for at least 30 minutes;
The supernatant was recovered and the volume was noted down and the pellet was discarded;
The supernatants from extractions I and II were combined and the combined volume was noted down;
The combined supernatants were filtered through 0.8-1.2 μm filters. The volume obtained after filtration was noted down.

C. Diafiltration
The diafiltration of the extract solution was performed in an ultrafiltration cassette membrane of 50 kDa. In this step, molecules having a molecular size of less than 50 kDa were removed;
The volume of distilled water necessary for the diafiltration was calculated according to the following formula:

ml filtrated extract×10=distilled water volume;
Once diafiltration was started, the pressure was fixed at 1.8 bar and the extract solution was maintained on ice and with constant agitation;
The conductivity of the diafiltrated extract was then checked to see whether it was less than 1050 μS/cm. If the conductivity was higher, diafiltration was continued with an additional volume of water;
The diafiltrated extract was filtrated through 0.22 μm filters;
The filtrated extract was then aliquoted in topaz crystal vials and then frozen to −80° C. The vials were maintained at −40° C. or less for a maximum period of 15 days;
The extract was then freeze dried.

The final product consists of a freeze-dried veterinary extract which is stored at 4° C. in freeze-dried conditions.

Immunochemical Characterisation

Protein Content

The protein content was determined by the Bradford method, following manufacturer's instructions.

Sodium Dodecyl Sulfate Polyacrylamide Gel Electrophoresis (SDS-PAGE)

Protein profiles were identified by SDS-PAGE under reducing conditions (samples incubated with 3-mercaptoethanol and heated for 10 minutes at 95° C.) in 2.67% C, 15% T acrylamide-bisacrylamide gels. Samples and Low Molecular Weight Standard (BioRad Laboratories, Hercules, Calif., USA) were run in the same gel. Gels were stained with 0.1% Coomassie Brilliant Blue R-250 (BioRad). Protein profiles were also analyzed with Any kD TGX gels (BioRad). Samples, HiMark prestained marker (LifeTechnologies, California, USA) and Broad Range Molecular Weight Standard (Bio-Rad) were run in the same gel.

2D

For 2-D electrophoresis, the extracts were purified and concentrated with a solution of ammonium sulphate in 2 separate steps until the saturation percentages of 40% and 80% were reached. Then, samples were centrifuged and the pellets were collected and reconstituted in ultrapure water. Concentrated extracts were washed using the ReadyPrep 2-D Cleanup Kit (BioRad) following the manufacturer's instructions. Proteins were separated according to their isoelectric point on ReadyStrip IPG strips (BioRad) with a pH range of 3 to 10, using Protean IEF Cell (BioRad). After the first dimension, the strips were equilibrated with ReadyPrep 2-D Kit buffers (BioRad) and proteins were separated in the second dimension according to their molecular weight. Gels were stained with Oriole fluorescent solution (BioRad) following the manufacturer's instructions.

Carbohydrate Content

The carbohydrate content determination is based on the procedure described in “Current Protocols in Food Analytical Chemistry E.1.1.1.-E.1.1.8, Eric Fourier (2001)”. Briefly, a standard curve of different glucose concentrations (0-1 mg/ml) and at least four dilutions starting from 4 mg/ml of the mite extracts were prepared. A volume of 0.5 ml of phenol 4% and 2.5 ml of H₂SO₄ were added to all the samples, vortexed and incubated for 20 minutes at room temperature. Then, absorbance was measured at 495 nm. Standard curve was obtained with glucose samples results and sample concentrations were interpolated to obtain the results.

Endotoxins Content

Endotoxins content (EU/ml) was determined by a colorimetric technique based on the Limulus Amebocyte Lysate assay and using an Endoscan V system (Charles River Laboratories). For this purpose, samples were dissolved at 1 mg/ml in free of endotoxins water and dilutions 1/100 and 1/500 were prepared.

Enzymatic Activity

Enzymatic activity of 19 different enzymes (described below) was evaluated in the D. farinae extracts with the APY-Zym System (Biomérieux) following manufacturer's instructions. Additionally, chitinase activity was analysed with a Chitinase Assay Kit (Sigma-Aldrich) following manufacturer's instructions.

Immuno-Blot

Electrophoretically separated proteins (by SDS-PAGE) were transferred to a PVDF (polyvinylidene difluoride) membrane (Trans-Blot® Turbo™ Transfer Pack, BioRad), blocked for 1 hour with 5% skimmed milk in phosphate buffer solution (PBS) 0.01 mol/L-Tween 0.1% and incubated overnight with sera from dogs positive to the allergen diluted in 0.01M PBS-0.1% Tween. Specific IgE binding to the extract was detected with peroxidase-conjugated antibodies, antidog-IgE-PO (Abd Serotec), developed with luminol solutions (Western Immun-Star™ Western C™ Kit, Bio-Rad) and detected by chemiluminescence (ChemiDoc XRS, Bio-Rad).

Major Allergen Quantification

Major allergens were quantified using ELISA sandwich method using enzyme-linked immunosorbent assay detection kits (Indoor Biotechnologies, VA, USA). Briefly, Nunc Maxisorp plates (Thermo Scientific, Waltham, Mass., USA) were coated with a specific monoclonal antibody diluted in carbonate/bicarbonate buffer (pH=9.6) and incubated overnight at 4° C. Afterwards, plates were blocked with BSA (bovine serum albumin) 1% in PBS 0.01 M-Tween 0.05%. Then, samples and standard were added in serial one half dilutions with BSA 1% in PBS 0.01 M-Tween 0.05%. Secondary specific monoclonal antibody (biotinylated) was added and streptavidin was finally used. Reaction with development solution (chromogen) was measured at optical density (OD) 450 nm after stopping with sulfuric acid. Standard curve was obtained using a 4-parameters logistic fit by the least-squares method, where sample concentrations were interpolated to obtain the results.

ELISA Assays

Specific IgE to D. farinae was measured in the pool of sera by direct ELISA. Briefly, microplates (Immulon IV; Thermo Scientific) were coated with same amount of protein of the D. farinae extracts and incubated overnight at room temperature. Plates were blocked for 1 h with 5% skimmed milk in PBS 0.01 mol/L-Tween 0.1%. The pool of sera was serially diluted and incubated for 2 h. After washing, the secondary antibody (diluted 1:10000) consisting on goat anti-dog IgE:HRP (horseradish peroxidase) was added. Finally, microplates were washed, the reaction developed and OD measured at 450 nm on an automated ELISA plate reader.

Size Exclusion Chromatography

The extracts were dissolved in 40 mM pH 7.4 phosphate buffer; NaCl 150 mM and filtered through 0.45 μm filters. One mg protein of the extracts was loaded into a Superdex 75 16/60 (GE Healthcare) column and analyzed in an AKTAexplorer system (GE Healthcare). The absorbance at 280 nm was registered during 120 minutes and the chromatograms analyzed with Unicorn software.

Allergens Identification

Bands were cut from an SDS-PAGE gel (Coomassie stained), reduced with DTT 10 mM, treated with iodoacetamide and digested with trypsin. Obtained peptides were analyzed by liquid chromatography coupled to a 5600 TRIPLE TOFF mass-spectrophotometer. Raw data were analyzed with MASCOT server against NCBI Acari database.

Relative Protein Quantification

Protein digestion: The extracts were resuspended in 50 mM NH₄HCO₃ (pH 8.5). Proteins were extracted by ultrasonic probe disgregation-solubilization, precipitated and the pellet was resuspended in 8 M urea/50 mM NH₄HCO₃ (pH 8.5). Proteins were reduced (DTT 20 mM) and alkylated (iodoacetamide 35 mM). Afterwards, the sample was digested with porcine trypsin, cleaned-up, dried-down and stored at −20° C. until the subsequent nanoUPLC-mass spectrometry analysis.

LC-MSMS analysis: The peptide mixtures were analyzed in a nanoAcquity liquid chromatographer (Waters) coupled to a LTQ-Orbitrap Velos (Thermo Scientific) mass spectrometer. Peptides were trapped on a Symmetry C18™ trap column (Waters), and were separated using a C18 reverse phase capillary column (Waters).

Data dependent analysis (DDA): Eluted peptides were subjected to electrospray ionization in an emitter needle (PicoTip™, New Objective). Peptide masses (m/z 300-1700) were analyzed in data dependent mode where a full Scan MS was acquired in the Orbitrap. Generated raw data files were collected with Thermo Xcalibur (v.2.2) and searched against Dermatophagoides farinae database.

Targeted Analysis: Acquired data and searches were evaluated in Skyline (v.3.1). Peptides identified with high confidence that proved to be stable over the course of the tests, were selected. Samples were acquired in triplicate with a Parallel Reaction Monitoring-Targeted MS/MS method for quantification purposes. Fibrinopeptide B standard (Glufib) (Waters) was added to each sample for normalization purposes.

Query samples (veterinary extract) were compared against Control samples (human extract) by utilizing statistical significance among the two conditions with a Student's T-test, with 95% confidence at peptide level.

ELISA Potency Assays

Briefly, microplates (Immulon IV; Thermo Scientific) were coated with the veterinary D. farinae extract (2 μg/well) and incubated overnight at room temperature. Plates were blocked for 1 h with 5% skimmed milk in PBS 0.01 mol/L; Tween 0.1%. For the inhibition assay, sera were preincubated with serial dilutions of the inhibitory extract in a Nunc plate (Thermo Scientific) for 2 hours before the addition to the Immulon IV. The pool of sera was incubated for 2 h. After washing, the secondary antibody (diluted 1:10000) consisting of goat anti dog IgE:HRP was added. Finally, microplates were washed, the reaction developed and optical density (OD) measured at 450 nm on an automated ELISA plate reader. The percentage of inhibition of the sera was represented respect to log μg extract for calculating the μg that produces the 50% of inhibition.

Cats Immunoblot

Veterinary and human D. farinae extracts were electrophoretically separated (by SDS-PAGE) and transferred to a PVDF (polyvinylidene difluoride) membrane (Trans-Blot® Turbo™ Transfer Pack, BioRad), blocked for 1 hour by drying the membrane at room temperature and incubated overnight with sera from cats diluted 1/6 in 0.01M PBS. Specific IgE binding to the extract was detected incubating the membrane 2 hours with biotinylated antibodies anti-feline-IgE (Greer), then incubated with streptavidin for 1 hour, developed with luminol solutions (Western Immun-Star™ Western C™ Kit, Bio-Rad) and detected by chemiluminescence (ChemiDoc XRS, Bio-Rad).

SDS-PAGE

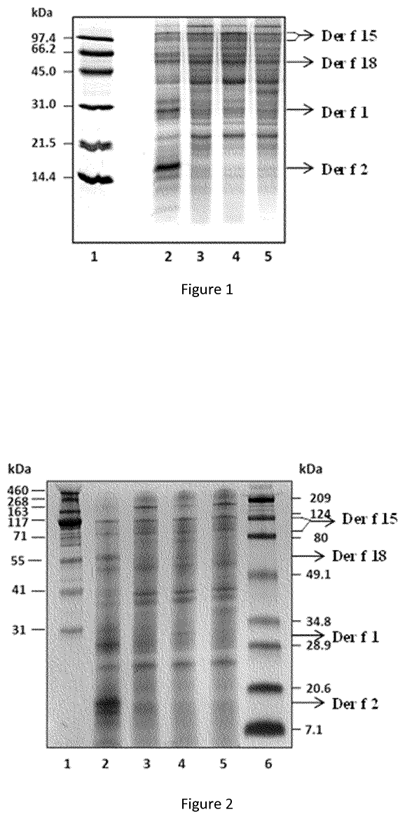

Ten μg protein of the lyophilized samples were analysed by SDS-PAGE under reducing conditions with 15% acrylamide gels (FIG. 1) and TGX commercial gels (Bio-Rad) (FIG. 2). Main allergens identified in the samples are indicated in the figures.

The same protein profile was observed (FIG. 1) in veterinary extracts 1, 2 and 3, with a significant reduction in Der f 2 band (about 15 kDa) and Der f 1 band (about 30 kDa) and an increase in intensity in proteins higher than 40 kDa with respect to the human extract. The band at about 20 kDa identified as ferritin in previous studies was also more intense in the three veterinary extracts with respect to the human extract.

Regarding the TGX gels (FIG. 2), the same protein profile was observed in extracts 1, 2 and 3. Moreover, the same differences in the relevant allergens between the human and veterinary extracts was also observed.

2D

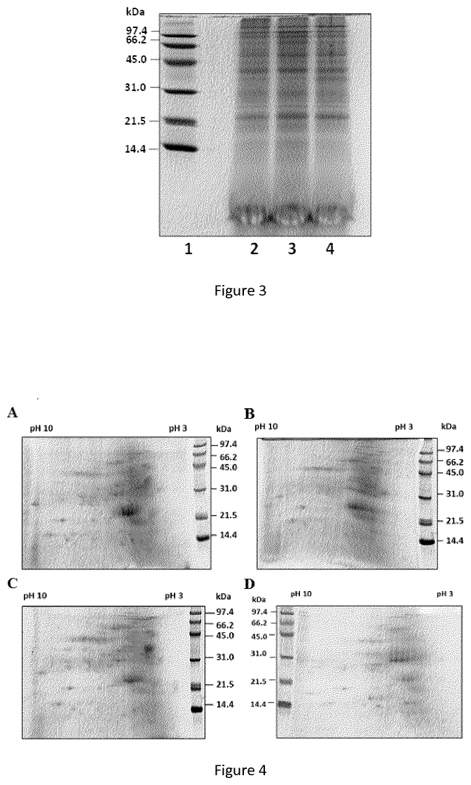

As an additional study on the analysis of the veterinary extracts' protein profile, a 2D electrophoresis was performed. The same quantity of protein of each extract was cleaned and analysed by SDS-PAGE (FIG. 3).

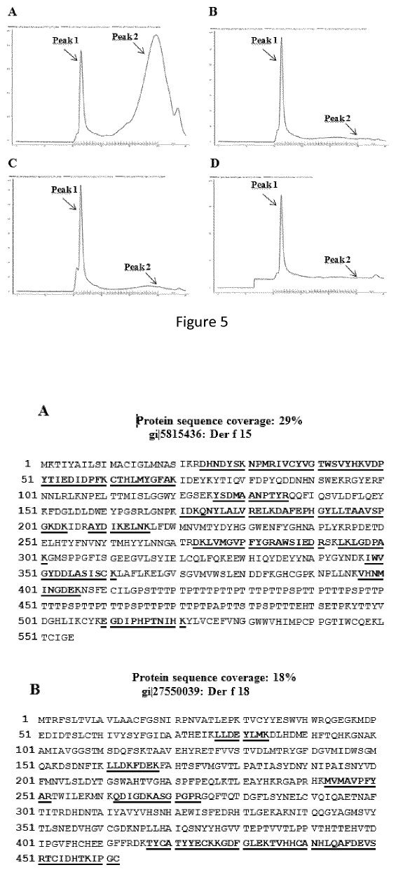

Then, 170 μg of each sample was submitted to the first dimension, the isoelectrofocusing. Next, all the extracts were subjected to the second dimension by SDS-PAGE and Oriole staining. An almost identical protein profile was obtained for the three veterinary extracts as can be observed in FIG. 4. A higher proportion of proteins between 20-100 kDa and at an acidic pH were observed in the veterinary extracts compared to the human extract (the 2D protein profile of the human extract was obtained from a previous study).

Size Exclusion Chromatography

Finally, as an additional analysis of the modification in the MW distribution of the veterinary extracts with respect to the human extract, all the samples were analysed by size exclusion chromatography. The same amount of protein of each extract (1 mg) was injected into a Superdex 75 16/60 column and analysed in an ÄKTA explorer system to obtain the MW distribution of each sample under non reducing conditions. The extracts were dissolved in phosphate buffer 40 mM pH 7.4 NaCl 150 mM and a volume of approximately 2 ml were injected into the column. The chromatographic profile obtained for all the extracts is shown in FIG. 5.

The two main chromatographic peaks were obtained for all the extracts, one at time 56 min approximately, and the second at time 100 min. However, when comparing the chromatographic profiles from veterinary extracts (FIG. 5B-D) to the one obtained for the human extract (FIG. 5A), it can be observed that the veterinary extracts presented a reduction in the 280 nm Abs signal at higher elution times (Peak 2), which correspond to lower MW proteins. On the other hand, Peak 1 containing high MW proteins was increased for the veterinary extracts compared to the human extract.

Major Allergens Quantification

Human major allergens Der f 1 and Der f 2 were quantified in the three veterinary extracts compared to the human extract. Mean content of both allergens in μg/mg lyophilised extract is shown in Table 1.

TABLE 1
Der f 1 and Der f 2 content (μg/mg ± SD) of human
extract and veterinary extracts (n = 2)

| | Der f 1 | Der f 2 |
|---|---|---|
| Human | 9.24 ± 1.4 | 20.95 ± 2.7 |
| Extract 1 | 3.20 ± 0.6 | 1.24 ± 0.2 |
| Extract 2 | 1.45 ± 0.3 | 0.95 ± 0.1 |
| Extract 3 | 1.45 ± 0.4 | 0.66 ± 0.0 |

Der f 1 was under 4 μg/mg in the three veterinary extracts and experienced a 4.5 times reduction on average with respect to the human extract. Der f 2 was found to be lower than 1.5 μg/mg in all the veterinary extracts which represented a 22 times reduction with respect to the human extract.

Carbohydrate Content

The carbohydrate content equivalent to glucose for all the extracts was determined by a spectrophotometric method with sulfuric acid and phenol as reagents. The results obtained in μg glucose/mg lyophilised extract are shown in Table 2.

TABLE 2
Glucose content (μg/mg ± SD) of human extract
and veterinary extracts (n = 2)

| | Glucose content |
|---|---|
| | μg/mg ± SD) |
| Human | 42.7 ± 0.2 |
| Extract 1 | 51.5 ± 7.7 |
| Extract 2 | 55.3 ± 5.0 |
| Extract 3 | 45.1 ± 11.7 |

The three veterinary extracts presented a very similar carbohydrate content compared to the human extract, although Extract 3 showed a higher variability between assays.

Endotoxins Content

The content of the extracts in endotoxins was determined. The four extracts were dissolved at a concentration of 1 mg/ml. Dilutions of 1/100 and 1/500 were used in each case. Table 3 shows the mean endotoxin content in EU/ml and EU/mg lyophilised extract obtained for each dilution and each extract.

TABLE 3
Endotoxin content of human and veterinary extracts (n = 2)

| | 1/100 | 1/500 | Mean | |
|---|---|---|---|---|
| | (EU/ml) | (EU/ml) | (EU/ml) | EU/mg |
| Human | 60.1 ± 0.3 | 54.6 ± 0.0 | 57.4 ± 3.9 | 57.4 ± 3.9 |
| Extract 1 | 64.9 ± 0.8 | 87.9 ± 0.5 | 76.4 ± 16.2 | 76.4 ± 16.2 |
| Extract 2 | 35 ± 0.8 | 34.8 ± 1.9 | 34.9 ± 0.1 | 34.9 ± 0.1 |
| Extract 3 | 83.4 ± 4.2 | 95.7 ± 6.0 | 89.5 ± 8.7 | 89.5 ± 8.7 |

Extract 1 presented a higher variability between the two dilutions tested. However, all the endotoxin contents values were within a similar range, between 30-90 EU/mg lyophilised extract, and the differences between the extracts could be due to the different raw material used for the production of each extract.

Enzymatic Activity

Most mite allergens have enzymatic activity, for that reason it is important to analyse the enzymatic activity of the extracts. The API-ZYM system was used to evaluate the activity of 19 different enzymes: alkaline phosphatase, esterase (C4), esterase lipase (C8), lipase (C14), leucine arylamidase, valine arylamidase, cystine arylamidase, trypsin, α-galactosidase, β-galactosidase, α-glucuronidase, α-glucosidase, β-glucosidase, N-acetyl-β-glucosaminidase, α-mannosidase, and α-fucosidase. Samples were prepared at 1 mg of protein/ml in distilled water. After incubation of each sample with the substrates, enzymatic activities were developed for 10 minutes. Results were interpreted optically according to the instructions manual ranging from 0 to 5 (0 means no colour, 5 means very strong colour). Results of 2 or below were considered negative. The semiquantitative determination results of the different enzymatic activities have been divided in groups depending on the kind of enzyme analysed.

Phosphatases (Table 4) were positive in all the extracts, being the most intense activity found for the acid phosphatase. No differences were found between human and veterinary extracts.

TABLE 4
Phosphatases activity of human and veterinary
extracts determined by APIZYM

| | Alkaline | Acid | Naphtol-AS-BI- |
|---|---|---|---|
| | phosphatase | phosphatase | phosphohidrolase |
| Human | 4 | 5 | 5 |
| Extract 1 | 4 | 5 | 4 |
| Extract 2 | 4 | 5 | 4 |
| Extract 3 | 4 | 5 | 4 |

Concerning proteases (Table 5), no cysteine arylamidase activity was found in any of the extracts whereas valine and leucine arylamidase were positive and similar in all the extracts. Trypsin activity was lower or even negative in veterinary extracts compared to the human extract and the α-chymotrypsin was positive in all the extracts (this is in contrast to the results obtained by Morales et al. (Enzymatic Activity of Allergenic House Dust and Storage Mite Extracts, J. Med. Entonol., 2013, 50, 147-54) which did not find α-chymotrypsin activity in a human D. farinae extract).

TABLE 5
Proteases activity of human and veterinary
extracts determined by APIZYM

| | Leucine | Valine | Cystine | | |
|---|---|---|---|---|---|
| | aryl- | aryl- | aryl- | | α- |
| | amidase | amidase | amidase | Trypsin | chymotrypsin |
| Human | 4 | 3 | 1 | 3 | 2 |
| Extract 1 | 4 | 3 | 0 | 1 | 2 |
| Extract 2 | 3 | 2 | 0 | 2 | 3 |
| Extract 3 | 4 | 2 | 0 | 0 | 3 |

Table 6 contains the information about lipases. No lipase activity was found in the extracts, whereas esterase and esterase lipase were positive and similar in all the extracts, with the exception of Extract 2 which was negative for the esterase.

TABLE 6
Lipases activity of human and veterinary
extracts determined by APIZYM

| | Esterase | Esterase lipase | Lipase |
|---|---|---|---|
| Human | 3 | 4 | 1 |
| Extract 1 | 2 | 4 | 0 |
| Extract 2 | 1 | 3 | 1 |
| Extract 3 | 3 | 4 | 1 |

Finally, Table 7 summarises glucosidase activity. This activity was evident in all the extracts, being positive for all the types of enzymes tested. No relevant differences were found between human and veterinary extracts, except for β-galactosidase and α-glucuronidase which presented an APIZYM level of 5 in all the veterinary extracts compared to the human extract (level 4).

TABLE 7
Glucosidases activity of human and veterinary extracts determined by APIZYM

| | α- | β- | α- | α- | β- | N-acetil-β- | α- | α- |
|---|---|---|---|---|---|---|---|---|
| | gal. | gal. | glucur. | glucos. | glucos. | glucosam. | mannos. | fucos. |
| Human | 4 | 4 | 4 | 4 | 4 | 3 | 4 | 4 |
| Extract 1 | 4 | 5 | 5 | 4 | 4 | 3 | 4 | 3 |
| Extract 2 | 4 | 5 | 5 | 5 | 5 | 2 | 4 | 4 |
| Extract 3 | 4 | 5 | 5 | 4 | 4 | 3 | 4 | 4 |

So, in general, it can be said that there were some differences between the human and the veterinary extracts regarding enzymatic activity. This result is explained by the loss of some types of proteins, many of them probably with enzymatic activity, in the veterinary extracts during the dialysis process. On the other hand, the three veterinary extracts presented very similar enzymatic activity profiles, demonstrating the high consistency of the product.

In addition to the APIZYM system, the chitinase activity of the extracts was evaluated with a chitinase assay kit (Sigma-Aldrich) following manufacturer's instructions. The kit is based on the enzymatic hydrolysis of chitinase substrates that releases p-nitrophenol that at a basic pH can be measured at 405 nm. The kit provides three different substrates for the detection of various types of chitinolytic activity. Samples were prepared at a concentration of 5 mg/ml for the test. Table 8 shows the chitinase activity for each sample and for each substrate hydrolysed.

TABLE 8
Chitinase activity of human and veterinary extracts for three different substrates (n = 2)

| | Substrate 1 (1 mg/ml) | | Substrate 2 (0.2 mg/ml) | | Substrate 3 (1 mg/ml) | |
|---|---|---|---|---|---|---|
| | mU/mg ext. | mU/mg prot. | mU/mg ext | mU/mg prot. | mU/mg ext | mU/mg prot. |
| Human | 20.6 ± 3.1 | 89.0 ± 13.3 | 10.7 ± 0.0 | 46.3 ± 0.0 | 11.2 ± 7.2 | 48.3 ± 31.2 |
| Extract 1 | 44.3 ± 6.7 | 92.7 ± 14.1 | 11.5 ± 0.7 | 24.1 ± 1.4 | 18.9 ± 11.2 | 39.5 ± 23.4 |
| Extract 2 | 57.6 ± 9.7 | 142.9 ± 24.1 | 11.7 ± 0.9 | 28.9 ± 2.3 | 21.7 ± 13.0 | 53.9 ± 32.2 |
| Extract 3 | 48.8 ± 9.2 | 104.7 ± 19.7 | 12.8 ± 2.3 | 27.5 ± 5.0 | 20.8 ± 11.9 | 44.6 ± 25.5 |

Substrate 1: 4-Nitrophenyl N-acetyl-β-D-glucosaminide; Substrate 2: 4-Nitrophenyl N, N′-diacetyl-β-D-chitobioside; Substrate 3: 4-Nitrophenyl β-D-N,N′,N″-triacetylchitotriose.

Regarding substrates 1 and 3, there were no relevant differences in mU/mg protein of chitinase activity between the extracts. For substrate 3, a high standard deviation was observed between assays, probably due to the different incubation times. Substrate 2, which is suitable for exochitinase activity detection, was higher in the human extract compared to the veterinary extracts. So despite the fact that Der f 15 and Der f 18 have been described as chitinases, no increase in chitinase activity was detected in the veterinary extracts.

Allergens Identification (Mass-Spectrometry Sequencing) In order to confirm the presence of the dog major allergens Der f 15 and Der f 18 in the three veterinary extracts the protein bands with the expected MW of 109/96 kDa (Der f 15) and 60 kDa (Der f 18) were cut from a SDS gel stained with Coomasie and sent to the Proteomics Unit of CSIC (“Centro Nacional de Biotecnología”, Madrid, Spain). Proteins were digested with trypsin and the obtained peptides were sequenced by mass-spectrometry and compared with the knowledge bases by MASCOT.

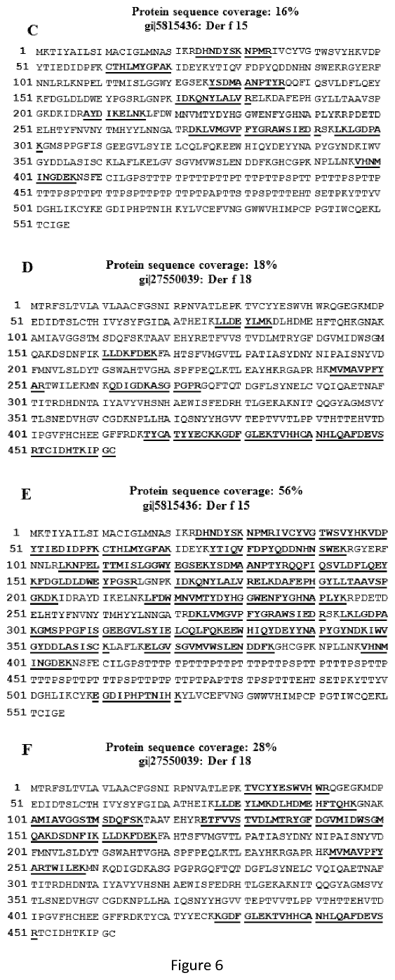

In FIG. 6, sequences obtained from Der f 15 (SEQ ID NO. 1) and Der f 18 (SEQ ID NO. 2) with the matched peptides are shown underlined, as well as the percentage of sequence coverage for each veterinary extract.

Relative Quantification of Allergens

In order to demonstrate that an increase in dog major allergens Der f 15 and Der f 18 is achieved in the veterinary extracts, a new methodology was developed for relative protein quantification by targeted proteomics (LC-MS/MS). This study was carried out by the Proteomics Unit of “Parc Cientific de Barcelona” (Barcelona, Spain).

To this purpose, the human extract was selected as a standard and the veterinary extract Extract 2 as a query sample. Both samples were digested with trypsin and the peptide solution obtained was analysed by nanoUPLC-mass spectrometry analysis according to the methodology described above. The raw data files obtained were searched against the Uniprot D. farinae database. Three peptides from Der f 15 and two from Der f 18 (Table 9) that proved to be stable over the course of the sets and did not present problems with detectability, degradation or non-optimal ionisation, were selected for the quantification assays. Peak areas of each peptide obtained in three different analyses were calculated and used for statistical analysis.

TABLE 9
Characteristics of the peptides acquired for the
quantification assays

| | | | Start | End | |
|---|---|---|---|---|---|
| Allergen | Sequence | m/z | time | time | SEQ ID NO. |
| Der f 15 | FDGLDLDWEYPGSR | 835.38 | 41 | 47 | 3 |
| Der f 15 | VDPYTIEDIDPFK | 776.38 | 41 | 47 | 4 |
| Der f 15 | IWVGYDDLASISCK | 813.89 | 37 | 43 | 5 |
| Der f 18 | TVHHCANHLQAFDEVSR | 505.99 | 18 | 24 | 6 |
| Der f 18 | GDFGLEK | 383.19 | 20 | 26 | 7 |

For Der f 15, there was a significant 2.97-fold increase, i.e. ×2.97, in the veterinary extract Extract 2 compared to human extract, whilst for Der f 18 there was a significant fold change of 0.24, i.e., ×0.24, in Extract 2 compared to the human extract.

These results confirm that veterinary extracts contain a higher content of Der f 15 allergen compared to a human D. farinae extract.

Immunoblot

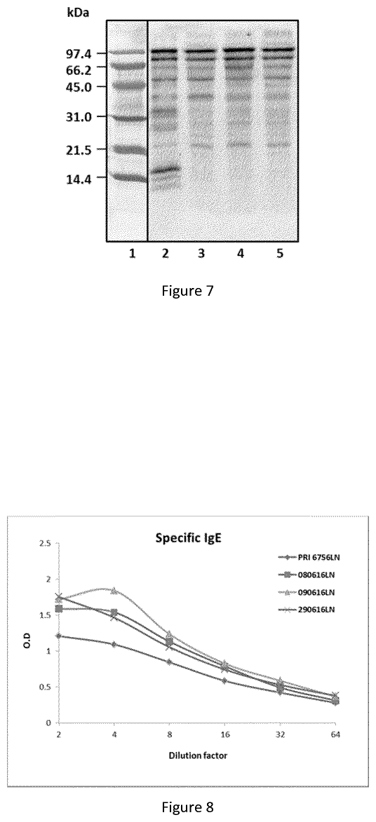

10 μg protein of each extract were analysed by immunoblot with a pool of sera (diluted 1/5) from dogs sensitised to this mite (FIG. 7). The immunoblot result confirmed the modification in the protein profile of recognition between the human and the veterinary extracts and highlighted the consistency between the three extracts.

Direct ELISA

All the extracts were also analysed by direct ELISA (8 μg protein of each extract/ml were used for coating the plate) with the same pool of sera. Higher specific IgE levels were found for the three veterinary extracts compared to the human extract (FIG. 8), which shows the higher affinity of the pool of sera from dogs with atopic dermatitis for the veterinary extracts.

### SUMMARY

The main results obtained from the production and characterization of three consistency extracts of the D. farinae are as follows:
The optimized ultrafiltration process using 50 k Pellicon® membranes and high pressure allowed the obtaining of three extracts of D. farinae with a high consistency as demonstrated by different analytical procedures;
The veterinary extracts had lower yields in the manufacturing process but a higher protein content with respect to the human extract;
The three extracts presented a modified protein profile with respect to the human extract, with a higher proportion of high MW proteins and a significant reduction in human major allergens, as demonstrated by electrophoresis, size exclusion chromatography and human major allergens quantification;
Concerning other extract characteristics, such as carbohydrate and endotoxins content, no relevant differences were found between human and veterinary extracts. There were slight differences in some enzymatic activities between human and veterinary extracts but regarding chitinase activity, there was no increase in this enzymatic activity in the veterinary extracts;
Dog major allergens Der f 15 and Der f 18 were identified in all the extracts by mass-spectrometry. Additionally, a new methodology which has not previously been used before for the characterization of the extracts has been optimized for relative protein quantification by targeted proteomics. This technique has allowed the demonstration with one of the veterinary extracts, that Der f 15 was significantly increased in this sample respect to the human extract;
From an immunological point of view, a pool of sera from dogs sensitized to this mite presented higher specific IgE levels to the three veterinary extracts compared to the human native extract.

Veterinary Dermatophagoides farinae extract standardization

1.1 In Vivo Study

A veterinary extract 4, prepared in the same manner as veterinary extracts 1 to 3 before, was used to prepare a solution for intradermal testing in dogs sensitized to D. farinae. For this purpose, a solution of the extract at a concentration of 1 mg/ml was prepared in SSFA (Saline phenolated solution with albumin), maintained under agitation for 2 hours, and filtrated with 0.22 μm filters. Together with veterinary extract 4, dogs were tested with histamine chlorhydrate 0.05 mg/ml (positive control) and SSFA as negative control.

Animals Included in the Study:

Fourteen dogs between 1 and 6 years old, with a clinical diagnosis of atopic dermatitis and presenting specific IgE against D. farinae>1000 ELISA Absorbance Units (EAU) (ELISA test from Greer Laboratories). Dogs with the following characteristics were excluded: dogs treated with corticoids, cyclosporine A, immunosuppressed compounds; dogs treated with immunotherapy; dogs suffering bacterial, viral or parasitic infections, immunodeficiency or immunopathology diseases.

A sample of 5 ml of serum was taken from each animal for in vitro analysis.

Intradermal Testing Procedure:

50 μl of the extract solution at 1 mg/ml or the controls were injected intradermally by duplicate in the thoracic area of the animals, previously shaved. After 15 minutes of the injection, the produced wheal was drawn and transferred to a paper.

Wheal Sizes Analysis:

The area of the wheals was measured with PC Draft (Microspot) software. Wheal sizes were considered positive if the area was >7 mm².

Results:

All the animals were positive to veterinary extract 4 with a mean wheal size of 115.3±66.8 mm².

1.2 In Vitro Study

Serum Samples Characterization

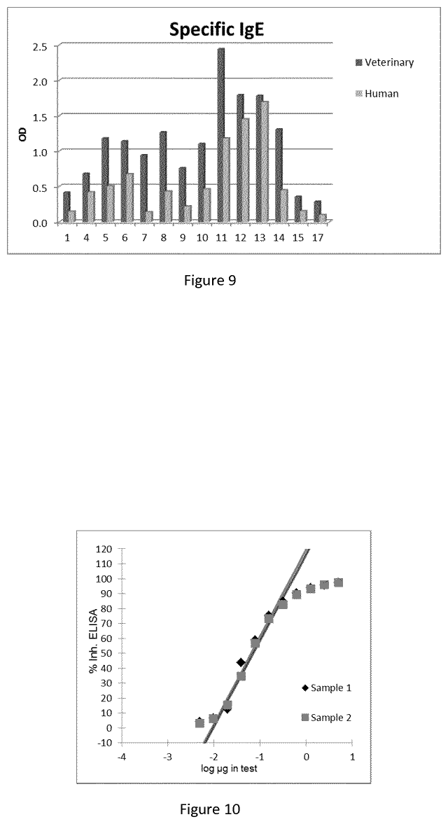

Specific IgE of each serum sample was analysed by direct ELISA (methodology has been previously described) against veterinary extract 4 and the human extract previously used. All serum samples presented specific IgE values significantly higher for veterinary extract 4 with respect to human D. farinae extract (FIG. 9).

Biological Potency

All the serum samples were used to prepare a pool to perform an in vitro standardization by ELISA potency assay. In Table 10 is shown the amount of veterinary extract 4 (μg) necessary to obtain 50% inhibition of the pool of sera from 5 different assays.

TABLE 10
Amount of veterinary extract 4 (μg) necessary to obtain 50%
of inhibition of the pool of dog sera from 5 different assays

| Assay | 50% inhibition (μg) |
|---|---|
| 1 | 0.063 |
| 2 | 0.069 |
| 3 | 0.053 |
| 4 | 0.061 |
| 5 | 0.065 |

The mean value obtained for 50% inhibition was 0.062±0.006 μg. An example of the curves obtained in this assay is shown in FIG. 10. 50% inhibition was calculated for a different batch of veterinary extract prepared as previously described (extract 5). The value obtained was 0.066 μg. This result demonstrates the consistency of different veterinary extract batches in terms of biological potency (IgE total allergenic activity).

Comparison of the Biological Potency of the Veterinary Extract with Respect to Human Extract

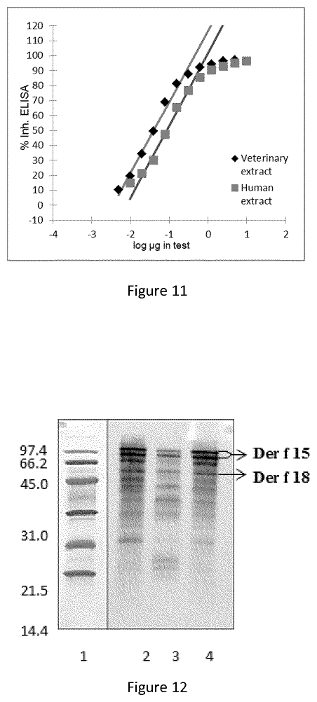

The ELISA potency assay previously described was used to analyze the potency of veterinary extract 4 with respect to the human extract. FIG. 11 shows the curves obtained in this assay.

The amount of human extract necessary for reaching 50% inhibition of a pool of sera of atopic dogs (selected in the same manner as described under “Veterinary Dermatophagoides farinae extract standardization”) was 0.132 μg, which represents a 2.13 fold increase with respect to veterinary extract 4 (0.062 μg), which demonstrates that veterinary extract 4 is approximately twice as potent relative to the human extract in terms of biological potency.

Immunoblot densitometry analysis for the estimation of Der f 15 and Der f 18 content The analysis of protein and allergenic profiles by densitometry can be used as an approach for the estimation of the amount of allergens in the extracts. For this reason, the band intensity of Der f 15 and Der f 18 allergens in allergenic profiles obtained with the pool of sera described in “Comparison of the biological potency of the veterinary extract with respect to human extract” was analyzed. Images corresponding to immunoblot experiments of different veterinary extracts and the human D. farinae extract were analyzed by ImageQuant software (GE Healthcare). The densitometry volume of each protein band as well as its molecular weight was analyzed and compared in the different samples.

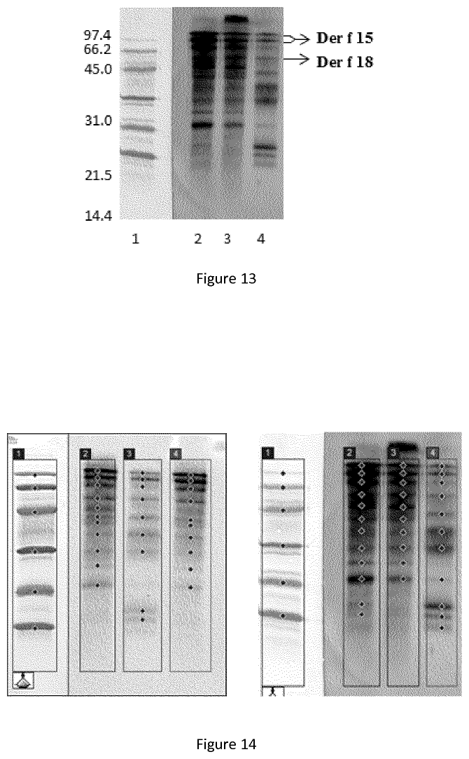

FIG. 12 and FIG. 13 show the immunoblot images analyzed by ImageQuant software showing the samples included in the analysis and major allergens Der f 15 and Der f 18. FIG. 14 shows the bands identified in both images by ImageQuant software. Values obtained for the different parameters analyzed and for each of the samples are shown in Tables 11 and 12.

TABLE 11
Volume and MW of each of the bands identified in image from
FIG. 12. Bands corresponding to Der f 15 (number 1 and 2)
and Der f 18 (band number 4) allergens are marked in bold.

| Low range MW standard | | | Veterinary extract 4 | | |
|---|---|---|---|---|---|
| Band | | MW | Band | | MW |
| No | Volume | (kd) | No | Volume | (kd) |
| 1 | 35464.00 | 97,000 | 1 | 60734.00 | 107.333 |
| 2 | 66797.00 | 66,000 | 2 | 72716.00 | 89.380 |
| 3 | 102555.00 | 45,000 | 3 | 67529.00 | 71.826 |
| 4 | 85339.00 | 30,000 | 4 | 47267.00 | 52.652 |
| 5 | 113878.00 | 20,100 | 5 | 75616.00 | 46.802 |
| 6 | 83895.00 | 14,400 | 6 | 40241.00 | 43.250 |
| | | | 7 | 32036.00 | 40.907 |
| | | | 8 | 25668.00 | 36.294 |
| | | | 9 | 31293.00 | 30.239 |
| | | | 10 | 13244.00 | 25.916 |
| | | | 11 | 29311.00 | 21.362 |

| Human extract | | | Veterinary extract 2 | | |
|---|---|---|---|---|---|
| 1 | 23348.00 | 102,167 | 1 | 66548.00 | 97,000 |
| 2 | 35043.00 | 85,642 | 2 | 80296.00 | 81,989 |
| 3 | 21474.00 | 68,799 | 3 | 70923.00 | 64,694 |
| 4 | 19169.00 | 52,037 | 4 | 38508.00 | 50,392 |
| 5 | 29530.00 | 42,957 | 5 | 35159.00 | 41,785 |
| 6 | 26585.00 | 36,013 | 6 | 15831.00 | 39,739 |
| 7 | 22801.00 | 30,000 | 7 | 24475.00 | 34,902 |
| 8 | 25769.00 | 16,947 | 8 | 16082.00 | 29,764 |
| 9 | 17174.00 | 15,653 | 9 | 10006.00 | 24,973 |
| | | | 10 | 14627.00 | 20,929 |

TABLE 12
Volume and MW of each of the bands identified in image from
FIG. 13. Bands corresponding to Der f 15 (number 1 and 2)
and Der f 18 (band number 4) allergens are marked in bold.

| Low range MW standard | | | Veterinary extract 5 | | |
|---|---|---|---|---|---|
| Band | | MW | Band | | MW |
| No | Volume | (kd) | No | Volume | (kd) |
| 1 | 6752.00 | 97,000 | 1 | 57421.00 | 114,222 |
| 2 | 25467.00 | 66,000 | 2 | 79715.00 | 97,000 |
| 3 | 49473.00 | 45,000 | 3 | 76210.00 | 76,755 |
| 4 | 42837.00 | 30,000 | 4 | 64094.00 | 56,191 |
| 5 | 50551.00 | 20,100 | 5 | 69077.00 | 47,490 |
| 6 | 65968.00 | 14,400 | 6 | 40960.00 | 40,819 |
| | | | 7 | 41925.00 | 35,647 |
| | | | 8 | 35178.00 | 29,203 |
| | | | 9 | 24296.00 | 24,721 |
| | | | 10 | 62490.00 | 20,924 |
| | | | 11 | 13603.00 | 16,275 |
| | | | 12 | 11024.00 | 14,647 |

| Veterinary extract 4 | | | Human extract | | |
|---|---|---|---|---|---|
| Band | | MW | Band | | MW |
| No | Volume | (kd) | No | Volume | (kd) |
| 1 | 48830.00 | 114,222 | 1 | 24884.00 | 112,500 |
| 2 | 60622.00 | 97,000 | 2 | 26198.00 | 95,090 |
| 3 | 54418.00 | 75,075 | 3 | 17742.00 | 75,075 |
| 4 | 45967.00 | 56,191 | 4 | 18943.00 | 54,551 |
| 5 | 50336.00 | 46,622 | 5 | 23501.00 | 43,060 |
| 6 | 24598.00 | 40,819 | 6 | 38645.00 | 35,324 |
| 7 | 27402.00 | 35,004 | 7 | 48094.00 | 29,203 |
| 8 | 24494.00 | 29,466 | 8 | 6976.00 | 20,756 |
| 9 | 12378.00 | 24,721 | 9 | 38710.00 | 15,893 |
| 10 | 26333.00 | 20,924 | 10 | 22113.00 | 14,264 |
| | | | 11 | 14696.00 | 12,364 |

Bands number 1 and 2 from tables 11 and 12 correspond to Der f 15 allergen in all samples whereas Der f 18 corresponds to band number 4. Tables 13 and 14 summarize the information corresponding to the volume (intensity) of these allergens and the fold-increase respect to human extract:

TABLE 13
Volume of Der f 15 and Der f 18 and fold-increase (in
parenthesis) relative to human extract (FIG. 12).

| | Human | Veterinary | Veterinary |
|---|---|---|---|
| Allergen | extract | extract 4 | extract 2 |
| Der f 15 | 58391 | 133450 (2.3) | 146844 (2.5) |
| Der f 18 | 16169 | 47267 (2.5) | 38508 (2.4) |

TABLE 14
Volume of Der f 15 and Der f 18 and fold-increase (in
parenthesis) respect to human extract (FIG. 13).

| | Human | Veterinary | Veterinary |
|---|---|---|---|
| Allergen | extract | extract 5 | extract 4 |
| Der f 15 | 51082 | 109452 (2.2) | 137136 (2.7) |
| Der f 18 | 18943 | 45967 (2.3) | 64094 (3.4) |

The volume of intensity showing the recognition of Der f 15 and Der f 18 allergens showed a high increase in veterinary extracts (between a 2.2 and 3.4 fold-increase, i.e. between ×2.2 and ×3.4 increase) relative to human extract. These results support the increase in the amount of these allergens in veterinary extract with respect to the D. farinae human extract.

Veterinary Extract for Allergy Treatment of Other Mammals: Cats

In order to evaluate if the veterinary extract could be used for the treatment of allergies in mammals other than dogs, the profile of sensitization of cats sensitized to D. farinae was analyzed.

Seven animals (1-7) positive to D. farinae (EAU>150; ELISA test from Greer Laboratories) and two negative controls (C1, C2) (EAU<150) were included in the study.

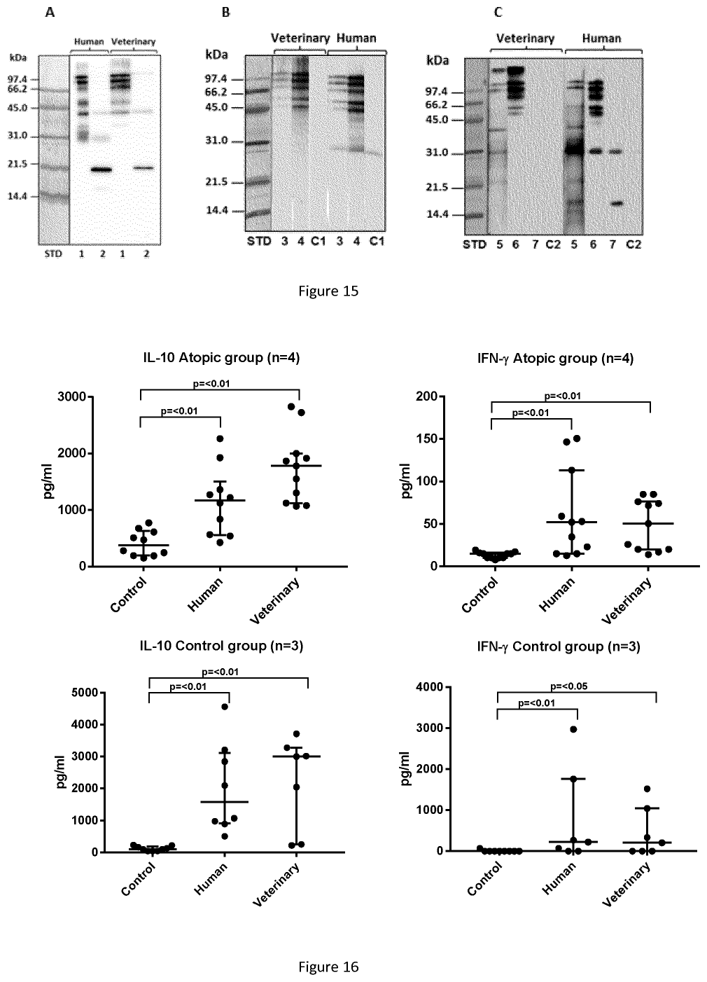

FIG. 15 shows the allergenic profile of the different animals included in the study. All positive animals (number 1-7) recognized bands with molecular weights from 14 to more than 100 kDa in veterinary extract 4 and/or the human extract, whereas negative controls (C1, C2) did not show any band of recognition.

Two main allergenic profiles were found among positive animals with clear differences in the MW of the recognized bands. Two animals recognized bands of low MW whereas several animals recognized mostly proteins higher than 40 kDa. Cat number 2 recognized mainly a 20 kDa-band and cat number 7 recognized bands of 30 and 14 kDa, that could correspond with human major allergens Der f 1 and Der f 2. On the contrary, cats number 1, 3, 4, and 6 mainly recognized proteins higher than 40 kDa, including dog major allergens Der f 15 and Der f 18. Finally, cat number 5 recognized proteins of high and low MW.

According to this information, those cats presenting an allergenic profile of bands higher than 40 kDa and including Der f 15 and Der f 18 allergens, could be treated with the veterinary mite allergen extract.

In-Vitro Cellular Assays

The objective of this study was to evaluate the capacity of the veterinary Dermatophagoides farinae extract 2 and human Dermatophagoides farinae extract for inducing cytokines production in Peripheral-Blood Mononuclear Cells (PBMCs) from atopic and non-atopic dogs.

1.1. Methodology

PBMCs culture supernatants were obtained from four atopic dogs and three healthy controls. The four atopic dogs presented a clinical history of atopic dermatitis and a specific IgE level against D. farinae>1000 EAU (ELISA test from Greer Laboratories). Dogs with the following characteristics were excluded: dogs treated with corticoids, cyclosporine A and immunosuppressed compounds; dogs treated with immunotherapy; dogs suffering bacterial, viral or parasitic infections, immunodeficiency or immunopathology diseases. The three healthy dogs did not present any clinical symptoms and had a specific IgE level against D. farinae<150 EAU.

Cytokine content in supernatants was measured by ELISA-based Milliplex® Mag Dog kit (Millipore) performed in accordance with the manufacturer's instructions. Briefly, PBMCs (2.5×10⁶ cells per well) from donors (four atopic and three non-atopic controls) were stimulated in triplicate with the human extract or veterinary extract 2 (40 μg protein/ml), and the production of IFN-γ and IL-10 was measured by duplicate in culture supernatants after 24 and 48 hours of incubation at 37° C. in 5% CO₂ atmosphere. Culture medium RPMI-1640 (Sigma-Aldrich) was used as negative control and Concanavalin A (Con A, 3 μg/ml) and LPS (lipopolysaccharides, 3 μg/ml) were used as positive controls.

For cytokines quantification, standard data were adjusted to a five-parameter logistic curve and U Mann-Whitney statistical analysis was performed. A p-value<0.05 was considered statistically significant.

1.2. Results

Cellular studies showed a similar induction of IFN-γ and IL-10 by the human and veterinary extracts in atopic and control dogs (FIG. 16). After 24 hours, the human and veterinary extracts induced significantly higher levels of IL-10 than negative control (median (ICI) of 1170 [559-1502] pg/ml for the human extract and 1748 [1122-1998] pg/ml for the veterinary extract). IFN-γ after 48 hours of incubation was also significantly higher than negative control in cells from atopic dogs treated with the human (52.1 [15-113.2] pg/ml) and veterinary extracts (50.4 [20-76.3] pg/ml).

Results from positive controls (ConA and LPS) were in all cases significantly higher than negative control (p<0.05), data not shown.

Atopic and non-atopic dogs were also compared. Higher levels of both cytokines were observed in non-atopic dogs with the human and veterinary extracts with respect to atopic dogs.

Successful allergen immunotherapy should be accompanied by induction of regulatory T cells (Treg) and shift from a Th2 response towards a Th1 response. Additionally, the success of immunotherapy has been related to the capacity of allergen vaccines to specifically stimulate production of IL-10 and IFN-γ, which are involved in the Treg and Th1 responses, respectively, and a reduction of IL-4. This study demonstrates the capacity of human and veterinary D. farinae extracts for inducing the production of IL-10 and IFN-γ in PBMCs from dogs sensitized to this mite, suggesting an induction of Th1 and Treg responses and therefore, a beneficial immune response that could lead to tolerance.

As the dogs included in the study were not previously treated with immunotherapy, it is normal to not observe, for the moment, a modification of the Th1 capacity of the dogs treated with the veterinary extract compared to those treated with the human extract. However the up-regulation of IL-10 demonstrates that the veterinary extract has the capacity to re-establish the equilibrium Th1-Th2. IFN-γ levels in dogs treated with the veterinary extract would be expected to be significantly increase compared to dogs treated with the human extract following immunotherapy treatment.

Non-atopic animals are in an immunological status in which there is a correct balance between the Th2/Th1 responses, and therefore, the profile of cytokines production is also different. Thus higher levels of IL-10 and IFN-γ in non-atopic dogs were observed because at a basal state they are producing more of these regulatory cytokines than atopic dogs, and after stimulation with the human and veterinary D. farinae extracts, production is further increased.

1. A veterinary mite allergen extract enriched with proteins with molecular weights greater than 50 kDa.

2. The veterinary mite allergen extract according to claim 1, wherein the extract is derived from Dermatophagoides farinae.

3. The veterinary mite allergen extract according to claim 1, wherein the veterinary mite allergen extract is enriched with the allergens Der f 15 and Der f 18 relative to human extract, and/or wherein the veterinary mite allergen extract has lower levels of allergens Der f 1 and Der f 2 relative to human extract.

4. The veterinary mite allergen extract according to claim 1, wherein in a 50% inhibition ELISA IgE assay, the veterinary mite allergen extract is at least 1.5 times more potent than human extract.

5. The veterinary mite allergen extract according to claim 1, wherein in an immunoblot densitometry analysis, the concentration of Der f 15 is at least 1.5 times higher than human extract, and/or wherein the concentration of Der f 18 is at least 1.5 times higher than human extract.

6. The veterinary mite allergen extract according to claim 1, wherein the veterinary mite allergen extract comprises recombinant Der f 15 and Der f 18, the recombinant Der f 15 and Der f 18 have at least 70% homology with non-recombinant Der f 15 and Der f 18 respectively, and the veterinary mite allergen extract up-regulates expression of IFN-γ and IL-10 in peripheral blood mononuclear cells.

7. A process for producing a veterinary mite allergen extract comprising:
a) contacting a source material comprising a mite allergen with an allergen extract agent to produce a mixture of allergens dissolved in liquid phase, and a solid phase comprising non-allergenic residue;
b) subjecting the mixture to a separation step to isolate the allergens dissolved in the liquid phase, to produce a crude allergen extract;
c) subjecting the crude allergen extract to a medium molecular fraction removal step to remove molecules having a size of less than 50 kDa and applying a pressure of 1.8 bar during the removal step;
d) carrying out step c) at 3-5° C. until the allergen extract has conductivity of below 1050 μS/cm and/or until a specific volume of distilled water has been used, to obtain an enriched allergen extract.

8. The process according to claim 7, wherein the mite allergen is Dermatophagoides farinae.

9. The process according to claim 7, wherein the separation step is centrifugation.

10. The process according to claim 7, wherein the medium molecular fraction removal step comprises an ultrafiltration step, a diafiltration step, a dialysis step, or filtration.

11. A veterinary mite allergen extract obtained by the process of claim 7.

12. The veterinary mite allergen extract according to claim 11, wherein the source material is Dermatophagoides farinae.

13. A method of treating a mite allergy, the method comprising:
applying the veterinary mite allergen extract according to claim 1, to an animal,
wherein the animal comprises serum IgE antibodies which in an immunoblot assay react with Dermatophagoides farinae allergens Der f 15 and Der f 18, or recombinant Der f 15 and Der f 18, the recombinant Der f 15 and Der f 18 having at least 70% homology with non-recombinant Der f 15 and Der f 18 respectively.

14. A pharmaceutical composition comprising the veterinary mite allergen extract according to claim 1.

15. A vaccine comprising the veterinary mite allergen extract according to claim 1.

16. The veterinary mite allergen extract according to claim 4, wherein in a 50% inhibition ELISA IgE assay, the veterinary mite allergen extract is 2.0-2.5 times more potent than human extract.

17. The veterinary mite allergen extract according to claim 5, wherein in the immunoblot densitometry analysis, the concentration of Der f 15 is 2.0-3.0 times higher than human extract and/or wherein the concentration of Der f 18 is 2.0-3.5 times higher than human extract.

18. The veterinary mite allergen extract according to claim 6, wherein the recombinant Der f 15 and Der f 18 have at least 80% homology with non-recombinant Der f 15 and Der f 18 respectively.

19. The method according to claim 13, wherein the animal is a dog or a cat, and wherein the recombinant Der f 15 and Der f 18 have at least 80% homology with non-recombinant Der f 15 and Der f 18 respectively.

20. The method according to claim 19, wherein the recombinant Der f 15 and Der f 18 have at least 90% homology with non-recombinant Der f 15 and Der f 18 respectively.
